(19) United States
(12) Patent Application Publication
(10) Pub. No.: US 2017/0072091 A1
(43) Pub. Date: Mar. 16, 2017

(54) HYDROGEL INCLUDING SURFACE-TREATED NANOFIBER AND PREPARATION METHOD THEREOF

(71) Applicant: POSTECH ACADEMY-INDUSTRY FOUNDATION, Pohang-si (KR)

(72) Inventors: Dong Soo HWANG, Pohang-si (KR); Dong Yeop OH, Busan (KR); Do Hoon LEE, Pohang-si (KR); Jaehyuk JUNG, Gwangju (KR)

(21) Appl. No.: 15/310,696

(22) PCT Filed: Apr. 14, 2015
(86) PCT No.: PCT/KR2015/003725

§ 371 Date: Nov. 11, 2016

(30) Foreign Application Priority Data
May 15, 2014 (KR) 10-2014-0058438
Apr. 13, 2015 (KR) 10-2015-0051619

(51) Int. Cl. A61L 24/00; A61L 24/08

(52) U.S. Cl. CPC A61L 24/0031; A61L 24/08; A61L 24/0015

(57) ABSTRACT

Provided are a bioadhesive hydrogel including surface-treated nanofibers, a preparation method thereof, and use of thereof. The hydrogel including surface-treated nanofibers provided in the present invention may have excellent bioadhesive strength, thereby being widely applied to a bioadhesive, a scaffold for tissue engineering, a carrier for drug delivery, etc.

### TECHNICAL FIELD

The present disclosure relates to a bioadhesive hydrogel including surface-treated nanofibers and a preparation method thereof, a bioadhesive composition including the bioadhesive hydrogel, and use thereof as a scaffold for tissue engineering and a carrier for drug delivery.

### BACKGROUND ART

A bioadhesive refers to a substance having an adhesive property to a variety of biological samples such as cell membranes, cell walls, lipids, proteins, DNAs, growth factors, cells, tissues, etc., and is applicable to various biomedical fields such as tissue adhesives, hemostats, scaffolds for tissue engineering, carriers for drug delivery, tissue augmentation, wound healing agents, anti-adhesive agents, etc.

Such bioadhesive is required to have strong adhesion and crosslinking abilities, and must maintain its function in a living body for a long period of time. Bioadhesives that are currently commercialized or practically used include cyanoacrylate instant adhesives, fibrin glue, gelatin glue, polyurethane-based adhesives, etc.

However, there are significant limitations that bioadhesives using synthetic polymers exhibit very weak adhesion ability under an aqueous environment of the living body, and the cyanoacrylate-based bioadhesives induce adverse effects such as immune responses in the human body, etc.

Further, although fibrin-based bioadhesives practically applied to patients induce fewer adverse effects such as immune responses in human body, they have very low adhesion ability, which is a limitation in applications, and their prices are also expensive.

In the case of gelatin bioadhesives, formalin or glutaraldehyde used as a crosslinking agent also causes a crosslinking reaction with proteins in the living body, leading to tissue toxicity. Polyurethane-based adhesives have a problem that aromatic diisocyanate used as a synthetic raw material exhibits cytotoxicity.

Accordingly, to overcome these problems, there is a demand for the development of ideal biomaterials having strong adhesion and crosslinking abilities on the surface of biological tissues containing water and showing minimal adverse effects in the living body.

### DISCLOSURE

### Technical Problem

An object of the present invention is to provide a bioadhesive hydrogel, including surface-treated nanofibers including a dihydroxyphenyl moiety, a trihydroxyphenyl moiety, or tannic acid which is covalently bound to the surface of chitin nanofibers or chitosan nanofibers.

Another object of the present invention is to provide a bioadhesive composition including the bioadhesive hydrogel.

Still another object of the present invention is to provide a scaffold for tissue engineering including the bioadhesive hydrogel.

Still another object of the present invention is to provide a carrier for drug delivery including the bioadhesive hydrogel.

Still another object of the present invention is to provide a preparation method of the bioadhesive hydrogel.

Still another object of the present invention is to provide a method of preparing the bioadhesive hydrogel, the method including the steps of preparing a hydrogel using chitin nanofibers or chitosan nanofibers; and performing surface-treatment of the nanofibers by treating the surface of the nanofibers included in the hydrogel with a surface treatment material of the following Chemical Formula 1.

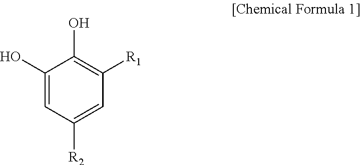

### Technical Solution

In order to achieve the above objects, an aspect of the present invention provides a hydrogel including surface-treated nanofibers including a dihydroxyphenyl moiety, a trihydroxyphenyl moiety, or tannic acid which is covalently bound to the surface of chitin nanofibers or chitosan nanofibers.

Another aspect of the present invention provides a preparation method of the hydrogel including surface-treated nanofibers.

### Best mode for Carrying out the Invention

A hydrogel including surface-treated nanofibers according to the present invention has a wound healing mechanism similar to that of Sea squirt. FIG. 6 is a schematic illustration showing the surface of Sea squirt. Sea squirt is covered by a tough covering, called tunic. The tunic is mainly composed of tunicin, cellulose nanofibrils (140 GPa, 26 nm×2.2 mm), and TOPA (3,4,5-trihydroxyphenyllalanine, pyrogallol amino acid) coated with protein microfibers. When Sea squirt gets hurt on the surface, the following two reactions occur for wound healing. First, when the wound is exposed to basic conditions (pH ˜8.2) of marine water, pyrogallol (a moiety of TOPA) oxidatively binds with tunicin or pyrogallol, and the pyrogallol group is bound coordinately with vanadium abundant in the blood of Sea squirt. During regeneration of tunic, covalent and non-covalent binding interactions are firmly formed and an elastic protective wall is formed to seal the edges of wound tissues and to control excessive bleeding.

Tannic acid is also called a sedimentary deposit or tannin, and widely found in plants such as the bark of tree, subterranean stems, fruits, etc. Tannic acid is a plant component including polyphenol and having a complex composition of tanning property. Tannic acid is widely distributed in the plant kingdom, although there is a concentration difference, and found in xylem, barks, leaves, fruits, roots, etc. Polyphenol is a subject of tanning action, and binds with proteins, particularly, collagen to convert the animal rawhide into stable leather. Tannic acid precipitates alkaloid, and binds with ferric irons to form a green or dark violet complex compound. Tannic acid is broadly divided into hydrolyzable tannin (I) and condensed tannin (II). Tannin (I) is ester of the hydroxyl group of sugars, quinic acid, etc., and the depside of gallic acid, hexahydroxydiphenic acid resulting from ring opening of ellagic acid, etc. Tannin (I) is hydrolyzed by acid, alkali, or tannase to produce gallic acid, ellagic acid, etc. Gallnut tannin (gall of Rhus chinensis) or fruit of Terminalia chebula has a high content of the tannin, and thus is used in leather treatment. Tannin (II) includes derivatives of flavanes such as catechins, leucoanthocyan, proanthocyan, stilbenes, etc. However, some of condensed forms have the properties of hydrolysable forms such as glucoside or ester of gallic acid, and when these condensed forms are polymerized by heating together with acids, red amorphous precipitates so-called phloba-pheneare are generated. Condensed tannin has an astringent taste (acerbic taste), and green tea includes ester of catechin and gallic acid at position 3 thereof, and astringent persimmons include leucoanthocyan. Gallic acids and ellagic acids are biosynthesized via the shikimic acid pathway and the skeletons of flavanes are biosynthesized via the combination of shikimic acid pathway and acetic acid-malonic acid pathway. Although the physiological roles of tannins in plants have not been clarified, their ability to strongly bind with proteins is considered to be correlated with some protection of the plants from diseases and insects.

Tannic acid is a type of natural polyphenols, and may have a structure of the following Chemical Formula. However, the molecular structure of tannic acid is not limited thereto, and tannic acid may exist in the forms of various polymers having different functional groups.

As shown in the following Chemical Formula, tannic acid has a large number of hydroxyl groups in the molecule, and therefore, has a characteristic of easily binding to large molecules such as polysaccharides, proteins, alkaloids, etc. In particular, three or more galloyl groups which may exist in the molecule form coordinate bonds with iron(III) ions to form a stable complex compound having an octahedral structure. Further, respective galloyl groups may form crosslinking with different iron (III) ions as central metals. The structure of tannic acid is as follows:

Structure of Tannic Acid

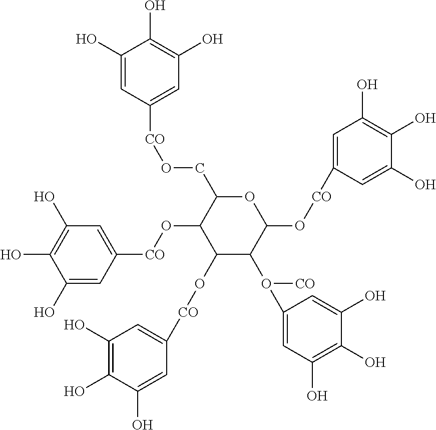

Hereinafter, the present invention will be described in more detail.

A bioadhesive hydrogel according to an embodiment of the present invention includes surface-treated nanofibers, and specifically, surface-treated nanofibers including a dihydroxyphenyl moiety, a trihydroxyphenyl moiety, or tannic acid which is covalently bound to the surface of chitin nanofibers or chitosan nanofibers.

The hydrogel is prepared by reacting tannic acid or a surface treatment material of the following Chemical Formula 1 with chitosan nanofibers, chitin nanofibers, or chitosan which is a tunicin-mimetic fiber structure to be covalently bound to the surface of the nanofibers.

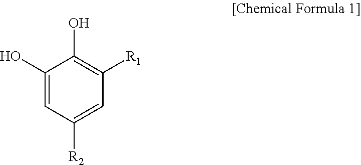

wherein R1 is a hydrogen atom or —OH,

R2 is —H, —COOH, —CHO, —NH₂, —SH, or an alkyl group prepared by substituting one or more hydrogen atoms of a straight or branched alkyl group having 1 to 10 carbon atoms or a circular alkyl group having 3 to 10 carbon atoms with a hydrogen atom, —COOH, —CHO, —NH₂, or —SH.

Further, the compound having Chemical Formula 1 may be selected from the group consisting of catechol, DOPA (3,4-dihydroxyphenylalanine), TOPA (3,4,5-trihydroxyphenyllalanine), pyrogallol, and gallic acid, and preferably, gallic acid and pyrogallol.

As the surface treatment binding method, a method capable of forming a covalent bond may be used. In Examples, a method of forming a peptide (—CONH—) bond between an amine group (—NH₂) of the nanofiber and a carboxyl group (—COOH) of gallic acid was used (FIG. 2A), but is not limited thereto.

Chitin, chitosan, tannin, and gallic acid used as basic materials for the reaction may be extracted from foods, but are not limited thereto.

The compound having Chemical Formula 1 may be used in an amount of 0.1 to 30% by weight, 0.1 to 10% by weight, 1 to 30% by weight, 10 to 30% by weight, or 10 to 20% by weight, based on 100% by weight of the chitin nanofibers, the chitosan nanofibers, or a mixture thereof.

As used herein, the “hydrogel” is formed by loss of fluidity of hydrosol due to cooling or by swelling of a hydrophilic polymer having a three dimensional network structure and a microcrystalline structure with water. The hydrogel refers to a material that retains a large amount of water in lattices of a polymer material to maintain the three dimensional structure, and takes a solid form even it is a liquid.

Hydrogel is able to absorb water of at least 20%, based on the total weight, and exists thermodynamically stable after being swollen in a solution, and has mechanical and physicochemical properties corresponding to the intermediate of liquid and solid. Hydrogel has a mechanical flexibility similar to that of actual tissues, and it includes a large amount of water, but binding of the gel is not easily broken by water. Therefore, hydrogel is actively applied to medical adhesives which are required to have adhesion with the wet living surface including water and also a resistance to the external water.

Therefore, the hydrogel having excellent tissue adhesive property according to the present invention may be applied to a variety of biomedical fields such as tissue adhesives, hemostats, scaffolds for tissue engineering, carriers for drug delivery, tissue augmentation, wound healing agents, anti-adhesive agents, etc. An embodiment of the present invention provides a surface-modified nanofiber hydrogel which may be used as an adhesive intended for medical purposes.

The hydrogel according to the present invention may have an adhesive strength of 5 to 100 Mpa, 5 to 80 Mpa, 20 to 100 Mpa, 20 to 80 Mpa, 50 to 80 Mpa, 50 to 60 Mpa, or 55 to 60 Mpa at a relative humidity of 50%. Further, the hydrogel may have an adhesive strength of 0.05 Mpa to 10 MPa, 0.1 to 10 Mpa, 1 to 10 Mpa, 5 to 10 Mpa, or 0.05 to 5 Mpa at a relative humidity of 100%.

Another aspect of the present invention provides a bioadhesive composition, including the hydrogel including the surface-treated nanofibers.

The bioadhesive composition of the present invention may be substituted for cyanoacrylate-based adhesives, fibrin-based adhesives, etc. which are mainly used at present, and may be applied to a variety of fields such as skins, blood vessels, digestive organs, cerebral nerves, plastic surgery, orthopedic surgery, etc.

For example, the biocompatible tissue adhesive of the present invention may be substituted for sutures for surgical operation, used for blocking unnecessary blood vessels, used for hemostasis and suturing of facial tissues, soft tissues such as cartilage, etc., hard tissues such as bones, teeth, etc., and also applied to household medicines.

Various application fields of the biocompatible bioadhesive composition of the present invention are summarized as follows:

In an embodiment, the bioadhesive of the present invention may be applied to internal and external surfaces of a living body, and that is, the bioadhesive of the present invention may be topically applied to the external surfaces of a living body such as skins, the surfaces of internal organs exposed during surgical operation, etc.

Further, the bioadhesive of the present invention may be used to adhere the damaged sites of tissues, to seal tissues for preventing leakage of air or fluid therefrom, to adhere a medical appliance to tissue, or to fill defects in tissues.

As used herein, the term “biological tissue” includes, is not particularly limited to, for example, skin, bone, neuron, axon, cartilage, blood vessel, cornea, muscle, fascia, brain, prostate, breast, endometrium, lung, pancreas, small intestine, liver, testes, ovaries, cervix, colon, stomach, lymph node, bone marrow, kidney, etc., but is not limited thereto.

In one embodiment, the bioadhesive of the present invention may be used for hemostasis and suture of soft tissues such as cartilage or hard tissues such as bone or tooth, and also applied to a precursor for bone component. The precursor component may be one or more selected from the group consisting of hydroxyapatite and octacalcium phosphate, but is not limited thereto.

In another embodiment, the bioadhesive of the present invention may be used for wound healing. For example, the biocompatible bioadhesive of the present invention may be used as a dressing applied to the wound.

In still another embodiment, the bioadhesive of the present invention may be used for skin closure. That is, the bioadhesive of the present invention may be topically applied for wound suturing, and may be substituted for suture threads. Further, the bioadhesive of the present invention may be applied to hernia repair surgery, and for example, applied to surface coating of meshes used in hernia repair.

In still another embodiment, the bioadhesive of the present invention may be used for suturing tubular structures such as blood vessels and for preventing leakage. Further, the bioadhesive of the present invention may be used for hemostasis.

In still another embodiment, the bioadhesive of the present invention may be used as an anti-adhesive agent after surgery. Adhesion occurs in all surgical sites, and is a phenomenon in which other tissues adhere to the wound surface around the surgical site. Adhesion occurs in about 97% after surgery, and particularly, severe problems are generated in 5 to 7% thereof. The adhesion may be prevented by minimizing the postoperative wound or by using an anti-inflammatory agent. Further, to prevent fibrosis, TPA (tissue plasminogen activator) may be activated or a physical barrier such as a crystalline solution, a polymer solution, a solid membrane, etc. may be used, but these methods may cause toxicity in a living body and also other adverse effects.

The bioadhesive of the present invention may be applied to a tissue exposed after surgery to prevent adhesion between the tissue and surrounding tissues.

In still another aspect, the present invention relates to a scaffold for tissue engineering including the hydrogel.

Tissue engineering refers to a process of culturing cells separated from a tissue of a patient on a scaffold to prepare a cell-scaffold complex, and then transplanting the prepared cell-scaffold complex into the body. Tissue engineering is applied to regeneration of almost all organs such as artificial skin, artificial bone, artificial cartilage, artificial cornea, artificial blood vessel, artificial muscle, etc.

The bioadhesive hydrogel of the present invention may provide a scaffold similar to a biological tissue, in order to optimize regeneration of the biological tissues and organs in tissue engineering.

Further, the scaffold of the present invention may be used to easily implement an artificial extracellular matrix, and utilized as a medical material such as cosmetics, wound dressing, dental matrix, etc.

To the hydrogel of the present invention, a variety of physiologically active substances involved in promoting cell growth and/or differentiation and helping regeneration and recovery of tissues via interaction with cells or tissues of a living body may be easily adhered.

In particular, since the amine functional group in the hydrogel may be used as a functional group for bioconjugation of other physiologically active substances, bioconjugation of physiologically active substances may be easily performed.

As used herein, the term “bioconjugation” refers to coupling of two or more biomolecules.

Further, the physiologically active substances generally refer to biomolecules which may be included in order to implement an artificial extracellular matrix having a similar structure to that of a natural extracellular matrix. The physiologically active substances may include cells, proteins, nucleic acids, sugars, enzymes, etc., and for example, cells, proteins, polypeptides, polysaccharides, monosaccharides, oligosaccharides, fatty acids, nucleic acids, etc., and particularly, cells.

The cells may be all cells including prokaryotic cells and eukaryotic cells, and exemplified by immunocytes and embryonic cells, including osteoblasts, fibroblasts, hepatocytes, neurons, cancer cells, B cells, white blood cells, etc.

In addition, the physiologically active substances may include a plasmid nucleic acid as a nucleic acid material, hyaluronic acid, heparin sulfate, chondroitin sulfate, or alginate as a sugar material, or a hormone protein as a protein material, but are not limited thereto.

In still another embodiment, the present invention relates to a carrier for drug delivery including the hydrogel.

The implantable tissue adhesive hydrogel according to the present invention may be used as an artificial extracellular matrix serving as a scaffold for drug delivery. The drug is not particularly limited, and may include chemicals, small molecules, peptides, protein drugs, nucleic acids, virus, antimicrobial agents, anticancer agents, anti-inflammatory agents, or a mixture thereof, but is not limited thereto.

The small molecules may include contrast agents (e.g., T1 contrast agents, T2 contrast agent such as supermagnetic materials, radioactive isotopes, etc.), fluorescent markers, or dyes, etc., but are not limited thereto.

The peptides or protein drugs may include hormones, hormone analogues, enzymes, enzyme inhibitors, signal transducing proteins or fragments thereof, antibodies or fragments thereof, single chain antibodies, binding proteins or binding domains thereof, antigens, adhesive proteins, structural proteins, regulatory proteins, toxin proteins, cytokines, transcription factors, blood coagulation factors, vaccines, etc., but are not limited thereto. More specifically, the peptides or protein drugs may include fibroblast growth factor (FGF), vascular endothelial growth factor (VEGF), transforming growth factor (TGF), bone morphogenetic protein (BMP), human growth hormone (hGH), porcine growth hormone (pGH), granulocyte-colony stimulating factor (G-CSF), erythropoietin (EPO), macrophage colony-stimulating factor (M-CSF), tumor necrosis factor (TNF), epidermal growth factor (EGF), platelet-derived growth factor (PDGF), interferons, interleukins, calcitonin, nerve growth factor (NGF), growth hormone releasing factor, angiotensin, luteinizing hormone releasing hormone (LHRH), luteinizing hormone releasing hormone agonist (LHRH agonist), insulin, thyrotropin-releasing hormone (TRH), angiostatin, endostatin, somatostatin, glucagon, endorphine, bacitracin, mergain, colistin, monoclonal antibodies, vaccines, or mixtures thereof, but are not limited thereto.

The nucleic acid may be RNA, DNA or cDNA, and a sequence of the nucleic acid may be a coding region sequence or a non-coding region sequence (e.g., antisense oligonucleotide or siRNA).

The virus may be a whole virus or a virus core including a nucleic acid thereof, that is, a nucleic acid of virus packaged without an envelope thereof. Examples of the virus or virus core to be transported may include Papillomvirus, Adenovirus, baculovirus, retrovirus core, Semliki virus core, etc., but are not limited thereto.

The antimicrobial agents may include minocycline, tetracycline, ofloxacin, fosfomycin, mergain, profloxacin, ampicillin, penicillin, doxycycline, thienamycin, cephalosporin, nocardicin, gentamicin, neomycin, kanamycin, paromomycin, micronomicin, amikacin, tobramycin, dibekacin, cefotaxime, cefaclor, erythromycine, ciprofloxacin, levofloxacin, enoxacin, vancomycin, imipenem, fusidic acid, and mixtures thereof, but are not limited thereto.

The anticancer agent may include paclitaxel, taxotere, adriamycin, endostatin, angiostatin, mitomycin, bleomycin, cisplatin, carboplatin, doxorubicin, daunorubicin, idarubicin, 5-fluorouracil, methotrexate, actinomycin-D, and mixtures thereof, but are not limited thereto.

The anti-inflammatory agents may include acetaminophen, aspirin, ibuprofen, diclofenac, indometacin, piroxicam, fenoprofen, flubiprofen, ketoprofen, naproxen, suprofen, loxoprofen, cinnoxicam, tenoxicam, and mixtures thereof, but are not limited thereto.

The present invention provides a bioadhesive hydrogel including surface-treated nanofibers including a dihydroxyphenyl moiety, a trihydroxyphenyl moiety, or tannic acid which is covalently bound to the surface of chitin nanofibers or chitosan nanofibers.

In an embodiment of the present invention, the hydrogel may be a hydrogel formed by binding between the dihydroxyphenyl moiety, the trihydroxyphenyl moiety, or the tannic acid included in the nanofibers and an oxidant or a metal ion.

More specifically, a method of preparing the bioadhesive hydrogel of the present invention includes the steps of preparing a hydrogel using chitin nanofibers or chitosan nanofibers; and performing surface-treatment of the nanofibers by treating the surface of the nanofibers included in the hydrogel with tannic acid or a surface treatment material of the following Chemical Formula 1:

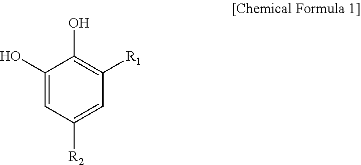

wherein R1 is a hydrogen atom or a hydroxyl group,

R2 is a hydrogen atom, —COOH, —CHO, —NH₂, —SH, or an alkyl group prepared by substituting one or more hydrogen atoms of a straight, branched, or circular alkyl group having 1 to 10 carbon atoms with one or more groups selected from the group consisting of hydrogen, —COOH, —CHO, —NH₂, or —SH.

In the method of preparing the hydrogel, the step of preparing the hydrogel may be performed by adding a metal ion or an oxidant to the nanofibers.

The metal ion may be Ca²⁺, Fe²⁺, Fe³⁺, V⁴⁺, V³⁺, V²⁺, etc., but is not limited thereto.

The oxidant may be sodium periodate, tetrabutylammonium periodate, hydrogen peroxide, etc., but is not limited thereto.

The addition amount of the metal ion or the oxidant may be 0.05 to 5 mol, 0.15 to 5 mol, 0.15 to 15 mol, 0.5 to 5 mol, 5 to 15 mol, 0.5 to 1.5 mol, or 0.15 to 1.5 mol, based on 1 mol of the dihydroxyphenyl moiety, the trihydroxyphenyl moiety, or the tannic acid included in the nanofibers.

The step of adding the metal is a step of coordinately bonding the dihydroxyphenyl moiety, the trihydroxyphenyl moiety, or the tannic acid included in the nanofibers to the metal ion.

In a specific embodiment, the present invention provides a method of preparing the bioadhesive hydrogel including the step of reacting the dihydroxyphenyl moiety, the trihydroxyphenyl moiety, or the tannic acid included in the nanofibers with Fe³⁻ ion.

Chitosan is a form obtained by removing acetamide groups from chitin, that is, with a degree of deacetylation of 50% or more, and the corresponding chitosan may be a chitosan with a degree of deacetylation of 85-95%. When the chitin nanofibers are used, the preparation method may be performed in a hydrogel state including the nanofibers, and when the chitosan nanofibers are used, the preparation method may be performed in a solution state where chitosan is dissolved, because chitosan is water-soluble.

Further, the step of adding the oxidant is a step of forming covalent bonds between the dihydroxyphenyl moiety, the trihydroxyphenyl moiety, or the tannic acid by oxidation of the dihydroxyphenyl moiety, the trihydroxyphenyl moiety, or the tannic acid included in the nanofibers with the oxidant.

In still another embodiment, a step of performing deacetylation treatment of the nanofibers may be additionally performed, before the step of preparing the hydrogel.

### Effect of the Invention

A hydrogel including surface-treated nanofibers of the present invention has excellent bioadhesion ability, and therefore, it is intended to provide a bioadhesive composition showing minimal adverse effects in a living body and a preparation method thereof. Accordingly, the bioadhesive hydrogel may be applied to various fields such as a bioadhesive, a scaffold for tissue engineering, a carrier for drug delivery, etc.

### BRIEF DESCRIPTION OF DRAWINGS

FIG. 1A is a TEM image of a hydrogel including chitin nanofibers prepared in the present invention;

FIG. 1B is an XRD image of the hydrogel including chitin nanofibers prepared in the present invention;

FIG. 2A is a structural formula according to a synthetic method of chitin nanofibers surface-treated with gallic acid according to the present invention;

FIG. 2B is a graph showing the results of measuring FT-IR functional groups of the hydrogel including chitin nanofibers according to the present invention and the hydrogel including chitin nanofibers surface-treated with gallic acid;

FIG. 2C is an image showing color development of Sea squirt flesh by treatment of Arnow assay reagent;

FIG. 3 is a graph showing the results of measuring adhesive strength of an adhesive composition including a chitin nanofiber hydrogel surface-treated with Fe³⁺ and gallic acid and an adhesive composition including a chitin nanofiber hydrogel surface-treated with NaIO₄ and gallic acid according to an exemplary embodiment to aluminum bars;

FIG. 4A is an illustration showing a binding mechanism that occurs between the chitin nanofiber hydrogel surface-treated with gallic acid and TOPA upon addition of an oxidant or Fe³⁺;

FIG. 4B is a graph showing the result of Raman spectroscopy of a binding reaction that substantially occurs between the chitin nanofiber hydrogel surface-treated with gallic acid and TOPA upon addition of Fe³⁺;

FIG. 5 is a graph showing the result of measuring cytotoxicity of the hydrogel including chitin nanofibers surface-treated with gallic acid in mouse osteoblastic cells (MC-3T3 e1);

FIG. 6 is a schematic illustration showing the surface of Sea squirt;

FIG. 7 is a schematic illustration showing the hydrogel including chitin nanofibers surface-treated with gallic acid;

FIG. 8 shows the result of 1H-NMR analysis to examine whether gallic acid is actually present in the chitosan hydrogel including gallic acid;

FIG. 9 is an image showing adhesive strength of two hydrogels according to the present invention;

FIG. 10 is an illustration showing a process of increasing adhesive strength by treating an oxidant to the chitosan hydrogel prepared according to an exemplary embodiment of the present invention; and

FIG. 11 is an image showing an adhesive strength test which was performed by shaking a hydroxyapatite/adhesive complex prepared according to an exemplary embodiment of the present invention in water.

### DETAILED DESCRIPTION OF THE EMBODIMENTS

The present invention may be variously modified and have various forms, and specific examples are exemplified and explained in detail in the following description. However, it is not intended to limit the present invention to the specific examples and it must be understood that the present invention includes every modifications, equivalents, or replacements included in the spirit and technical scope of the present invention.

Materials used in the following Examples, chitin, CaCl₂2H₂O (calcium chloride dehydrate), and gallic acid were purchased from Sigma-Aldrich (US), and EDC (1-Ethyl-3-(3-dimethylaminopropyl carbodiimide) and NHS (N-hydroxysuccinimide) were purchased from Thermo scientific.

### EXAMPLE 1

### Preparation of Chitin Nanofiber Hydrogel

1-1: Preparation of Hydrogel including Chitin Nanofibers

In order to prepare a chitin nanofiber-based hydrogel, a method of H. Tamura et al. (Tamura, H., Nagahama, H. & Tokura, S. Preparation of chitin hydrogel under mild conditions. Cellulose 13, 357-364 (2006)) was employed.

First, 850 g of CaCl₂ H₂O was dissolved in 1 L of methanol, and 20 g of chitin was added thereto, followed by mixing at 150° C. for 6 hours. Thereafter, to precipitate the chitin nanofiber hydrogel, 20 L of distilled water was added to 1 L of the methanol solution containing chitin nanofibers. Then, calcium ions were removed by water-based dialysis (MWCO=1,000).

For deacetylation of the surface of the chitin nanofibers included in the hydrogel, mercerization was performed. Specifically, the chitin nanofiber hydrogel was refluxed in NaOH (20 wt %) for 6 hours to deacetylate the surface, and the surface-deacetylated chitin nanofibers were precipitated by centrifugation at 10,000 rpm and 4° C. for 30 minutes, and the resulting precipitate was washed with water several times.

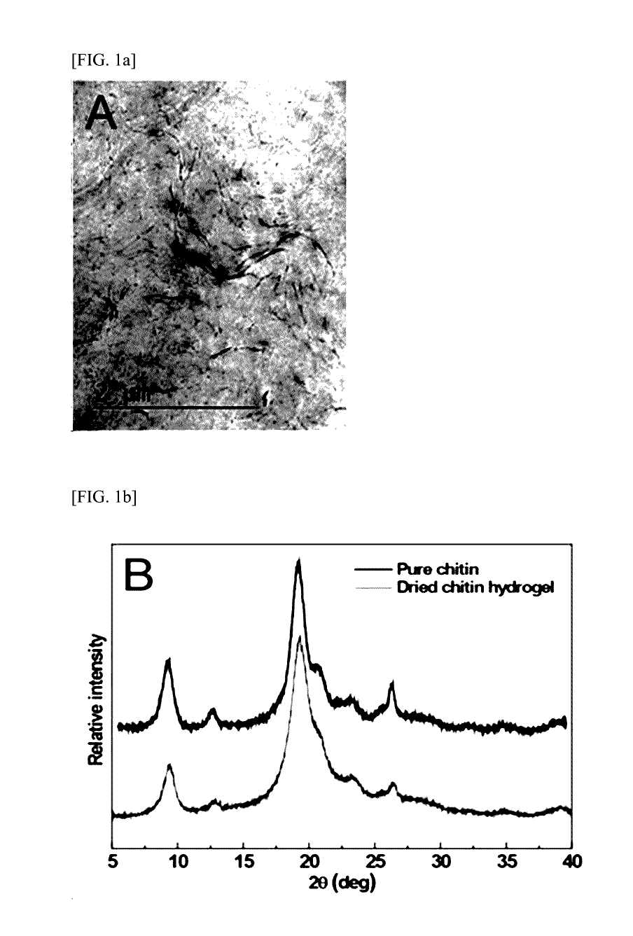

The hydrogel including chitin nanofibers thus obtained was analyzed by TEM (transmission electron microscope) and XRD (X-Ray Diffraction), and the results are given in FIGS. 1A and 1B.

The shape of the hydrogel including dried chitin nanofibers was studied by a high-resolution scanning electron microscope, JEOL JSM-7401F (SEM, Japan).

As shown in FIG. 1A, the nanofibers in the prepared chitin nanofiber hydrogel were identified by TEM. As shown in FIG. 1B, XRD analysis was performed to confirm whether the nanofibers were identical to the chitin nanofibers.

The XRD analysis was performed under conditions of 40 kV/100 mA and Ni-filtered Cu K a radiation using D/MAX-2500/PC (Rigaku, Japan), and XRD patterns were recorded from a diffraction angle of 5° to 40° at a scan speed of 4°/min.

1-2: Preparation of Hydrogel Including Chitin Nanofibers Surface-Treated with Gallic Acid

A hydrogel based on chitin nanofibers surface-treated with gallic acid was prepared, referring to the following literatures (Pasanphan, W., Buettner, G. R. & Chirachanchai, S. Chitosan conjugated with deoxycholic acid and gallic acid: A novel biopolymer-based additive antioxidant for polyethylene. Journal of Applied Polymer Science 109, 38-46 (2008); Yu, S. H. et al. Preparation and characterization of radical and pH-responsive chitosan-gallic acid conjugate drug carriers. Carbohydrate Polymers 84, 794-802 (2011); Pasanphan, W. & Chirachanchai, S. Conjugation of gallic acid onto chitosan: An approach for green and water-based antioxidant. Carbohydrate Polymers 72, 169-177,(2008).)

10 g of the chitin nanofiber hydrogel prepared in Example 1-1 were added to 50 ml of PBS buffer. 2 equivalents of gallic acid with respect to chitin monomer (acetyl-glucosamine) and 1.1 equivalents of EDC with respect to gallic acid were dissolved in 20 ml of methanol. At 15 minutes after completely dissolving EDC, NHS (1 equivalent of EDC) was added to the methanol solution. At 30 minutes after adding NHS, the buffer containing chitin hydrogel and the solution containing gallic acid/EDC/NHS were mixed with each other. The mixture was maintained for 12 hours. This procedure was performed in an ice bath, and EDC and NHS in the hydrogel synthetic product were removed by large-scale dilution, centrifugation, decantation, and water-based dialysis.

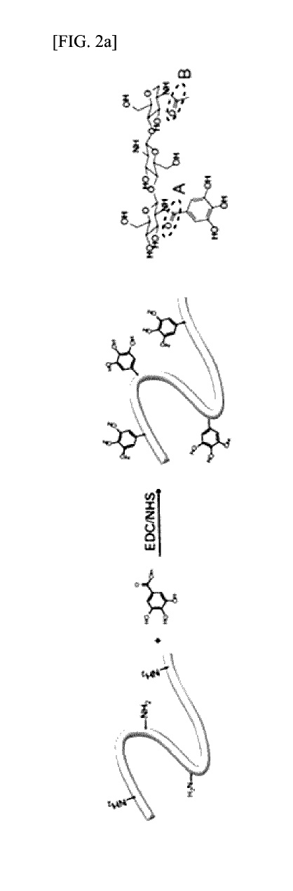

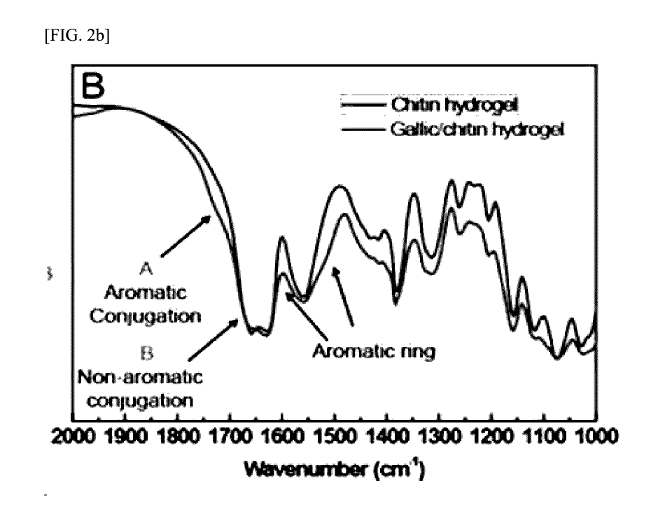

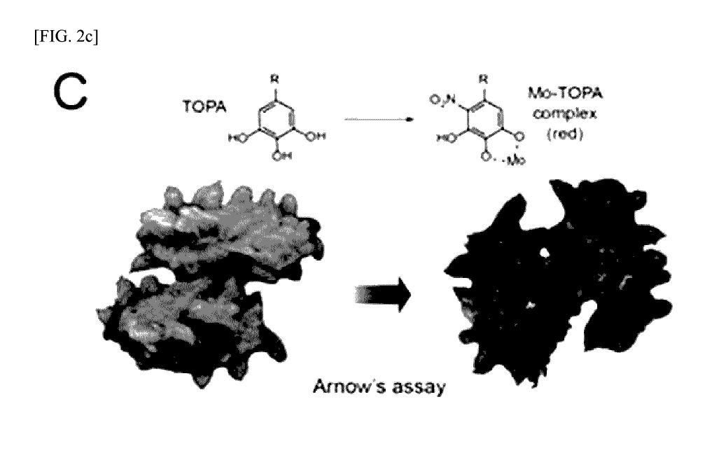

Gallic acid-chitin conjugation present in the hydrogel including chitin nanofibers surface-treated with gallic acid was analyzed by FT-IR (Fourier transform infrared spectroscopy), and the results are given in FIG. 2B.

As shown in FIG. 2B, the presence of gallic acid in the hydrogel including chitin nanofibers surface-treated with gallic acid was confirmed by FT-IR.

FT-IR analysis was performed using a single-beam MIDAC M 1200 (Midiac corporation, MA, USA) having a resolution of 4 cm⁻¹ and a range of 1000 to 4000 cm⁻¹.

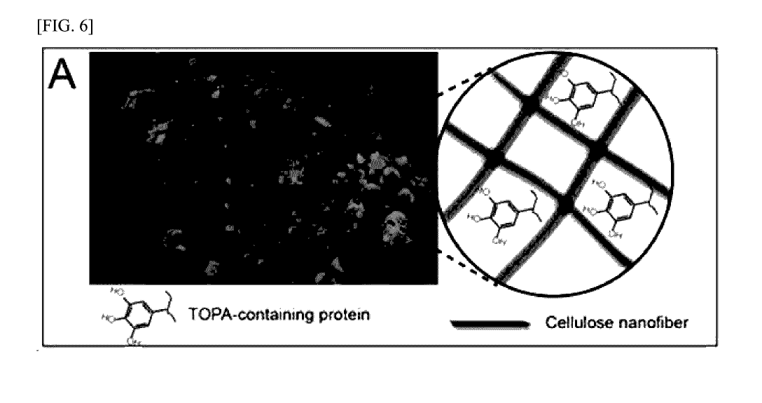

FIG. 6B is a schematic illustration showing the hydrogel including chitin nanofibers surface-treated with gallic acid.

### EXAMPLE 2

### Investigation of Gallic Acid Binding by Colorimetric Method

To quantify the content of gallic acid in the adhesive, a modified Arnow assay was performed together with a chitin hydrolysis method which is widely used in the analysis of amino acids and polyphenol extracts. 5 mg, 10 mg, and 15 mg of gallic acid and 25 mg of dried adhesive were sealed in a glass ampoule containing 500 μl of 6 M HCl and 20 μl of phenol (for minimal oxidation) under vacuum, respectively. The glass ampoules were heated to 110° C. 24 hours later, the solution in each glass ampoule was diluted 10-fold. 500 μl of 1.45 M sodium nitrite/0.4 M sodium molybdate solution was added to 500 μl of each diluted solution, and color changes were observed. 1 ml of 1M NaOH was added to each diluted solution, and color changes were observed.

The color changes of each diluted solution are shown in FIG. 2C.

As shown in FIG. 2C, when 500 μl of 1.45 M sodium nitrite/0.4 M sodium molybdate solution was added, the color was changed to dark yellow, and when 1 ml of 1M NaOH was added, the color was changed to dark red.

### EXAMPLE 3

### Preparation of Adhesive Composition

In order to examine adhesive strength of chitin nanofibers surface-treated with gallic acid (pyrogallol acid) on the surface similar to a biological tissue, different adhesion tests were performed. A curing process is required in order to maintain adhesive strength in water of the hydrogel including gallic acid-conjugated chitin nanofibers prepared in Example 1. Different curing methods by pyrogallol, that is, a curing method by coordinate bonding with metal ions and a curing method by covalent bonding between moieties were performed to prepare two kinds of adhesive compositions.

As the first method by coordinate bonding, pyrogallol-conjugated chitin nanofibers prepared in Example 1 was mixed with FeCl₃ at a molar ratio of pyrogallol:Fe³⁺ of 3:1, and cured by pyrogallol-Fe coordinate bonding to prepare an adhesive composition including the chitin nanofiber hydrogel surface-treated with Fe³⁺ and gallic acid.

As the second method by covalent bonding, pyrogallol-conjugated chitin nanofibers prepared in Example 1 was treated with an antioxidant NaIO₄ at a molar ratio of pyrogallol:IO₄⁻ of 2:1, and crosslinked by covalent bonding to prepare two kinds of adhesive compositions including the chitin nanofiber hydrogel surface-treated with IO₄⁻ and gallic acid.

Fe³⁺-DOPA hydrogel is known to have a strong reversible bond in water due to coordinate bonding between Fe³⁺ and catechol (a moiety of DOPA), and it is known that the coordinate bonding between a moiety of DOPA, catechol and Fe³⁺ can be analyzed by Raman spectroscopy (Holten-Andersen, N. et al. pH-induced metal-ligand cross-links inspired by mussel yield self-healing polymer networks with near-covalent elastic moduli. Proceedings of the National Academy of Sciences of the United States of America 108, 2651-2655 (2011)) (Kim, B. J. (2014). Mussel-Mimetic Protein-Based Adhesive Hydrogel. Biomacromolecules.). On the basis of these literatures, the coordinate bonding between pyrogallol group and Fe³⁺ was demonstrated by Raman spectroscopy. As a result of the Raman spectroscopy, it was found that the pyrogallol group of gallic acid forms a strong reversible bond with Fe³⁺.

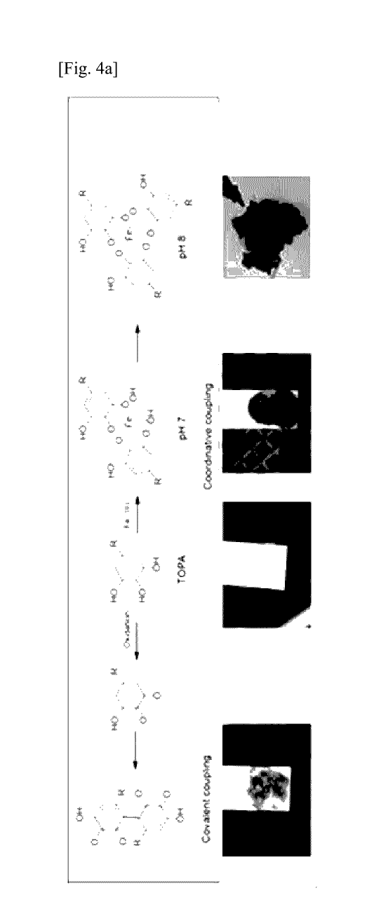

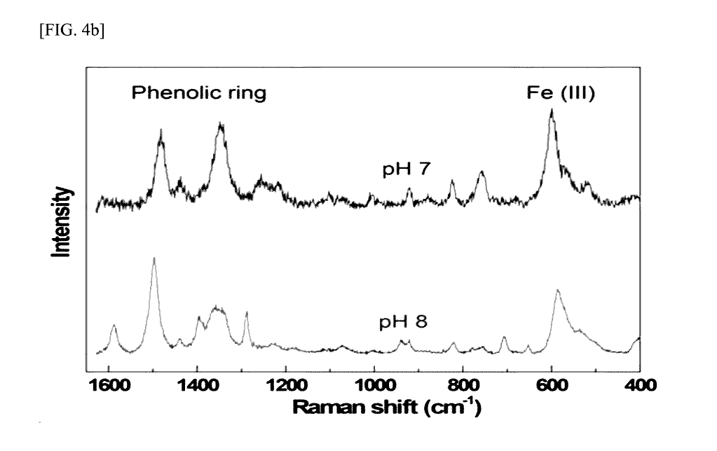

The results are shown in FIG. 4B. Raman spectra were obtained by using LabRam ARAMIS (Horiba Jobin-Yvon, France) under the following conditions. All spectra were obtained by collecting data in the range from 400 cm⁻¹ to 1600 cm⁻¹ by irradiating a sample with light at 785 nm.

As shown in FIG. 4B, chitin nanofibers surface-treated with Fe³⁺ and gallic acid form a coordinate bond, like Fe³⁺-DOPA complex, thereby forming a strong reversible bond in water, resulting in successful formation of a complex compound.

### EXAMPLE 4

### Measurement of Adhesive Strength in Water (Shear Strength) of Adhesive Composition

Adhesive strength in water of the two kinds of adhesive compositions of Example 3 was measured.

The composition was applied to 10 mm×10 mm area at the end of each of two aluminium bars having a size of 10 mm×50 mm. The ends of the two aluminium bars of 10 mm×10 mm, to which the adhesive composition was applied, were faced each other, and fixed with a clip. A pair of adhesion test samples was prepared by fixing the two aluminium bars with a clip. In this way, 5 pairs of samples were prepared for the two adhesive compositions, respectively, and then immersed in PBS (pH 7.4) buffer for 2 hours.

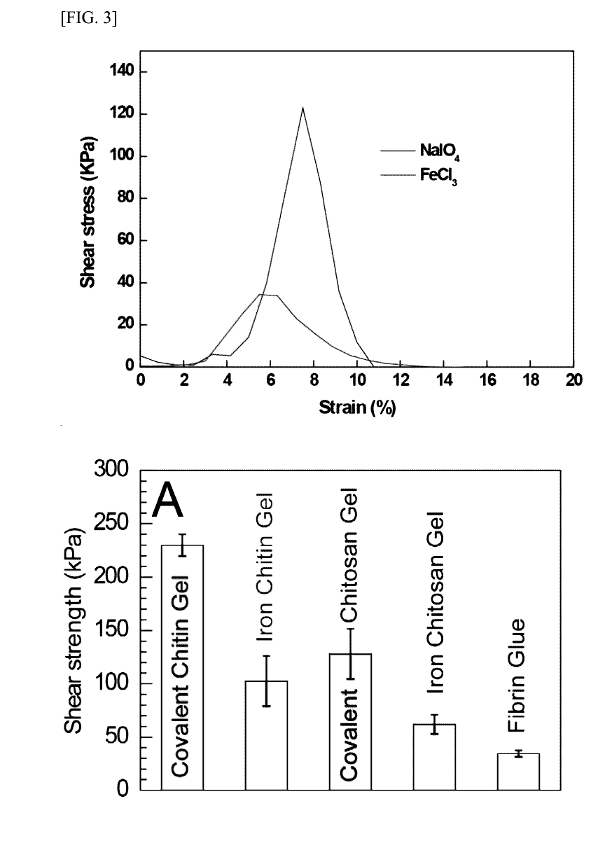

Adhesive strength in water was measured by the following method. Both ends of the aluminium bars were pulled using a universal testing machine (INSTRON) at a speed of 5 mm/min, and a force according to a distance was measured. Stress is F(force)/A(area) and is a value obtained by dividing the pulling force by unit area (10 mm×10 mm), and its unit is N/m². Strain means an extension ratio, and represented by [(final length−initial length)/initial length]×100. In a strain-stress curve, x axis represents strain and y axis represents stress. The highest stress value before detachment of the adhesive between the two aluminum bars is defined as an adhesive strength (shear strength) of the sample. The adhesive strength and the stress-strain curve of the two adhesive compositions are shown in FIG. 3. Reference literature (Kim, B. J. (2014). Mussel-Mimetic Protein-Based Adhesive Hydrogel. Biomacromolecules)

### EXAMPLE 5

### Test of Cytotoxicity on Osteoblasts

In order to examine whether the adhesive compositions of Example 3 have cytotoxicity, a cell viability test of osteoblasts (Mouse osteoblst MC3T3-e1; Riken cell bank) was performed. An experimental method and a principle of measuring cell viability (number of cells) are as follows.

A cell culture dish was coated with chitin nanofibers surface-treated with gallic acid which is the adhesive composition, and used as an experimental group. A non-coated cell culture dish was used as a control group. A solution in which osteoblasts were uniformly dispersed in advance was seeded in these two kinds of cell culture dishes. For 3 days, the number of osteoblasts growing in the different cell culture dishes was monitored using CCK-8 (cell counting kit-8). Since a CCK-8 reagent reacts with a cell metabolite to develop color, the number of cells can be determined by absorbance of a medium including cells. Further, an increase in the number of cells is proportional to cell viability, thereby quantifying cell viability.

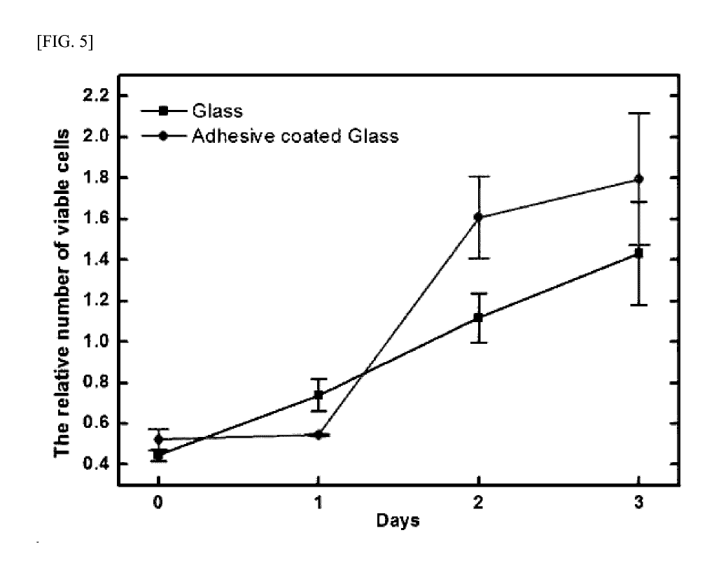

The results of the cell viability test are shown in FIG. 5. The red circles indicate the results of cell viability on a glass substrate which was treated with the adhesive composition of the present invention, and the black circles indicate the results of cell viability on a glass substrate which was not treated with the adhesive composition of the present invention as a control group.

As shown in FIG. 5, cell viability on the glass substrate which was treated with the adhesive composition of the present invention was rather improved, compared to the control group, suggesting that the chitin nanofiber hydrogel surface-treated with gallic acid prepared in the present invention is non-toxic to a living body.

### EXAMPLE 6

### Preparation of Chitosan Nanofiber Hydrogel

A chitosan hydrogel including gallic acid was prepared, referring to the following literatures (Pasanphan, W., Buettner, G. R. & Chirachanchai, S. Chitosan conjugated with deoxycholic acid and gallic acid: A novel biopolymer-based additive antioxidant for polyethylene. Journal of Applied Polymer Science 109, 38-46 (2008); Yu, S. H. et al. Preparation and characterization of radical and pH-responsive chitosan-gallic acid conjugate drug carriers. Carbohydrate Polymers 84, 794-802 (2011); Pasanphan, W. & Chirachanchai, S. Conjugation of gallic acid onto chitosan: An approach for green and water-based antioxidant. Carbohydrate Polymers 72, 169-177,(2008).)

1 g of chitosan (chitosan with a degree of deacetylation of 85-95%) was dissolved in 100 ml of a hydrochloric acid solution at pH 2, and pH of the chitosan solution was increased to pH 5.5 by slowly dropping 1 M sodium hydroxide solution thereto. 2 equivalents of gallic acid with respect to a monomer (glucosamine) and 1.1 equivalents of EDC with respect to gallic acid were dissolved in 20 ml of methanol. At 15 minutes after completely dissolving EDC, NHS (1 equivalent of EDC) was added to the methanol solution. At 30 minutes after adding NHS, the chitosan solution and the solution containing gallic acid/EDC/NHS were mixed with each other. The mixture was maintained for 12 hours. This procedure was performed in an ice bath, and EDC and NHS in the hydrogel synthetic product were removed by large-scale dilution, centrifugation, decantation, and water-based dialysis.

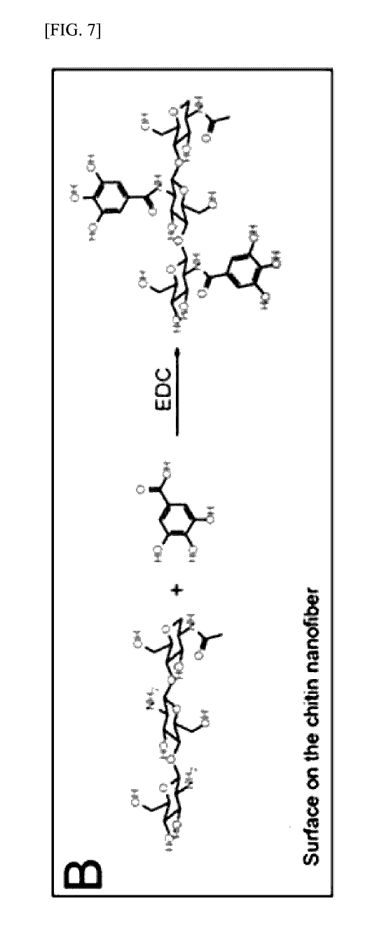

Gallic acid-chitosan conjugation present in the chitosan hydrogel including gallic acid was analyzed proton nuclear magnetic resonance (¹H-NMR), and ¹H-NMR data of chitosan hydrogel including gallic acid are given in FIG. 7.

As shown in FIG. 7, the presence of gallic acid in the chitosan hydrogel including gallic acid was confirmed by ¹H-NMR.

### EXAMPLE 7

### Preparation of Adhesive Composition using Chitosan Hydrogel

A curing process is required in order to maintain adhesive strength in water of the chitosan hydrogel including gallic acid. Different curing methods by pyrogallol, that is, a curing method by coordinate bonding with metal ions and a curing method by covalent bonding between moieties were performed to prepare two kinds of adhesive compositions. The method of preparing the adhesive composition is substantially identical to that of Example 3, except that the chitosan hydrogel including gallic acid of Example 6 was used in this Example, instead of the chitin hydrogel including gallic acid of Example 3.

As the first curing method by coordinate bonding with metal ions, the chitosan hydrogel including gallic acid prepared in Example 6 was mixed with FeCl₃ at a molar ratio of pyrogallol:Fe³⁺ of 3:1, and cured by pyrogallol-Fe coordinate bonding to prepare an adhesive composition including the chitosan hydrogel including Fe³⁺ and gallic acid.

As the second curing method by covalent bonding between moieties, the chitosan hydrogel including gallic acid prepared in Example 6 was treated with an antioxidant NaIO₄ at a molar ratio of pyrogallol:IO₄⁻ of 2:1, and crosslinked by covalent bonding to prepare two kinds of adhesive compositions including the chitosan hydrogel including IO₄⁻ and gallic acid.

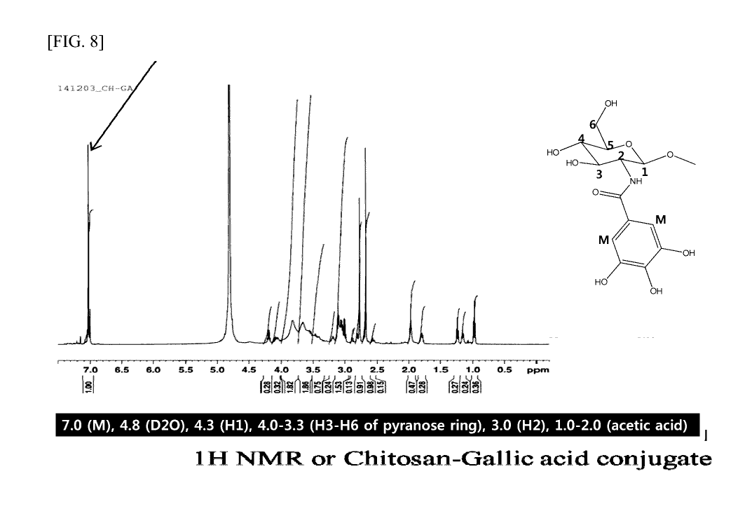

An image of the prepared two kinds of adhesive compositions is shown in FIG. 8. The image of FIG. 8 shows adhesive strength of the prepared hydrogel, and shows the hydrogel crosslinked by covalent bonding between pyrogallol moieties and the hydrogel crosslinked by coordinate bonding with metals. In this regard, as pH increases, the number of coordinate bonds can be maximized by pH, and therefore, the image was obtained by increasing pH. That is, the image simply shows the shape of the hydrogel. Further, the results of investigating physical properties upon using chitosan nanofibers of the present invention are shown in FIG. 3.

### EXAMPLE 8

### Characterization of Bioadhesive Including Chitosan Hydrogel

An image showing hydroxyapatite adhered to the chitosan hydrogel in water and a brief experimental method, and mechanical strength of the chitosan hydrogel was not measured. However, in order to perform the clinical operation by adhesion to the mucous membrane in water, the chitosan/gallic acid hydrogel was used as an adhesive material for fixing a bone conductive material such as hydroxyapatite (calcium-phosphate complex) practically used for bone regeneration upon clinical surgery or bovine bone powder. Characterization of the adhesive to be clinically used under an aqueous environment in the body was performed by an experimental method as follows.

The chitosan/gallic acid prepared in Example 7 was dissolved in water at a concentration of 20% (w/w) to form a hydrogel including chitosan. In this regard, the concentration of chitosan/gallic acid is not limited to 20% (w/w), and it was confirmed that sufficient adhesive strength was maintained at a concentration of 10% (w/w) or higher.

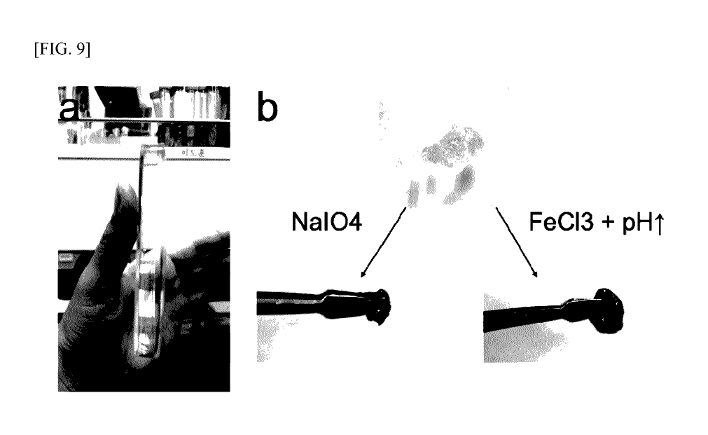

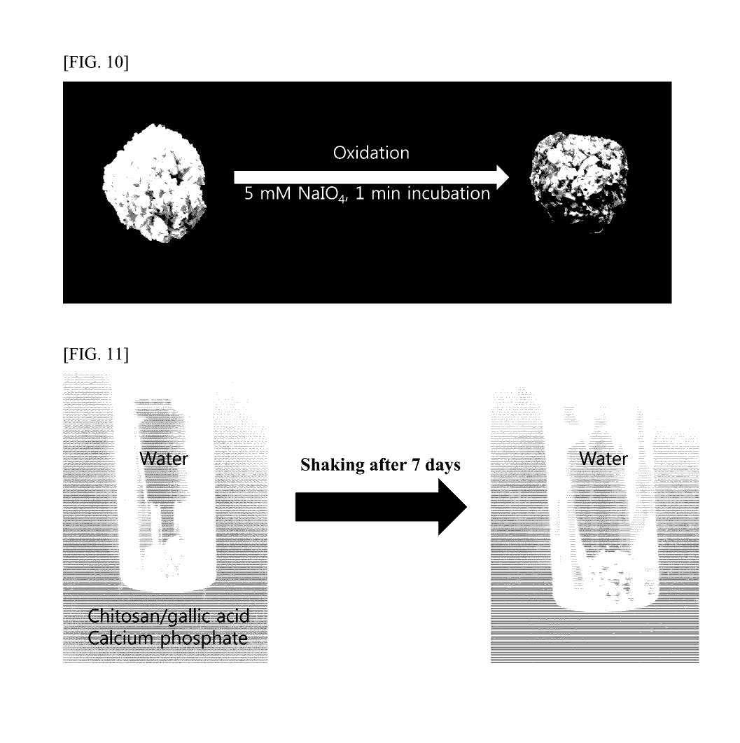

The prepared hydrogel was mixed with hydroxyapatite or bovine bone powder, and a strong covalent bond was formed by using an oxidant NaIO₄ to increase adhesive strength, as shown in FIG. 9A. Even though no oxidant was used, sufficient adhesive strength was obtained, however, in the present Example, the oxidant was used in order to induce strong binding.

Further, the prepared hydroxyapatite/adhesive complex was put in water, followed by vigorously shaking. As a result, no detachment was observed, and strong adhesive strength was maintained for 1 week or longer, as shown in FIG. 9B.

This Example suggests that the corresponding adhesive has a potential as a bioadhesive, and thus has applicability to a sealant used in mucosal tissues and organs in the body or a carrier for sustained drug delivery.

1. A bioadhesive hydrogel comprising chitin nanofibers or chitosan nanofibers, wherein the nanofibers are surface-treated nanofibers comprising a dihydroxyphenyl moiety, a trihydroxyphenyl moiety, or tannic acid covalently bound to the surface thereof.

2. The bioadhesive hydrogel of claim 1, wherein the surface-treated nanofibers are obtained by treating tannic acid or a surface treatment material having the following Chemical Formula 1 to chitin nanofibers, chitosan nanofibers, or chitosan: 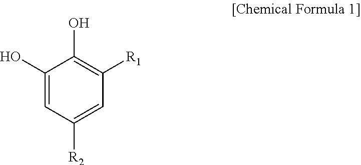
wherein R1 is a hydrogen atom or a hydroxyl group,
R2 is a hydrogen atom, —COOH, —CHO, —NH₂, —SH, or a straight, branched, or circular alkyl group having 1 to 10 carbon atoms, wherein the alkyl group is each independently substituted with one or more groups selected from the group consisting of hydrogen, —COOH, —CHO, —NH₂, and —SH.

3. The bioadhesive hydrogel of claim 2, wherein the surface treatment material having Chemical Formula 1 is selected from the group consisting of gallic acid, pyrogallol, catechol, DOPA (3,4-dihydroxyphenylalanine), TOPA (3,4,5-trihydroxyphenyllalanine), and pyrogallol.

4. The bioadhesive hydrogel of claim 2, wherein the surface treatment material is comprised in an amount of 0.1 to 30% by weight, based on 100% by weight of the nanofibers.

5. The bioadhesive hydrogel of claim 1, wherein the hydrogel has an adhesive strength of 5 to 100 Mpa at a relative humidity of 50%, and an adhesive strength of 0.05 Mpa to 10 MPa at a relative humidity of 100%.

6. The bioadhesive hydrogel of claim 1, wherein the hydrogel is bioconjugated with a physiologically active substance.

7. The bioadhesive hydrogel of claim 6, wherein the physiologically active substance is a cell, a protein, a nucleic acid, a sugar, an enzyme, or a mixture thereof.

8. The bioadhesive hydrogel of claim 1, wherein the hydrogel is formed by bonding between the dihydroxyphenyl moiety, the trihydroxyphenyl moiety, or the tannic acid bound to the chitin nanofibers or the chitosan nanofibers and metal ions by adding the metal ions.

9. The bioadhesive hydrogel of claim 8, wherein the metal ion is Ca²⁺, Fe²⁺, Fe³⁺, V⁴⁺, V³⁺, or V²⁺.

10. The bioadhesive hydrogel of claim 8, wherein the metal ion is added in an amount of 0.05 to 5 mol, based on 1 mol of the dihydroxyphenyl moiety, the trihydroxyphenyl moiety, or the tannic acid comprised in the nanofibers.

11. The bioadhesive hydrogel of claim 1, wherein the hydrogel is formed by covalent bonding between the dihydroxyphenyl moiety, the trihydroxyphenyl moiety, or the tannic acid comprised in the chitin nanofibers or the chitosan nanofibers by adding an oxidant.

12. The bioadhesive hydrogel of claim 11, wherein the oxidant is sodium periodate, tetrabutylammonium periodate, or hydrogen peroxide.

13. The bioadhesive hydrogel of claim 11, wherein the oxidant is added in an amount of 0.05 to 5 mol, based on 1 mol of the dihydroxyphenyl moiety, the trihydroxyphenyl moiety, or the tannic acid comprised in the nanofibers.

14. A bioadhesive composition comprising the bioadhesive hydrogel of claim 1.

15. The bioadhesive composition of claim 14, wherein the bioadhesive composition is applied to one or more biological tissues selected from the group consisting of skin, bone, neuron, axon, cartilage, blood vessel, cornea, muscle, fascia, brain, prostate, breast, endometrium, lung, pancreas, small intestine, liver, testes, ovaries, cervix, colon, stomach, lymph node, bone marrow, and kidney.

16. The bioadhesive composition of claim 14, wherein the bioadhesive composition is applied to one or more bone precursors selected from the group consisting of hydroxyapatite and octacalcium phosphate.

17. A scaffold for tissue engineering, comprising the bioadhesive hydrogel of claim 1.

18. A carrier for drug delivery, comprising the bioadhesive hydrogel of claim 1.

19. A method of preparing a bioadhesive hydrogel, comprising the steps of preparing a hydrogel using chitin nanofibers or chitosan nanofibers; and
performing surface-treatment of the nanofibers by treating the surface of the nanofibers comprised in the hydrogel with tannic acid or a surface treatment material of the following Chemical Formula 1: 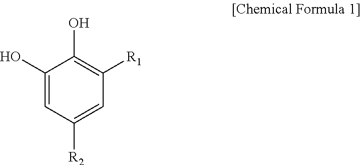
wherein R1 is a hydrogen atom or a hydroxyl group,
R2 is a hydrogen atom, —COOH, —CHO, —NH₂, —SH, or a straight, branched, or circular alkyl group having 1 to 10 carbon atoms, wherein the alkyl group is each independently substituted with one or more groups selected from the group consisting of hydrogen, —COOH, —CHO, —NH₂, and —SH.

20. The method of claim 19, wherein the step of preparing the hydrogel is performed by adding a metal ion or an oxidant to the nanofiber.

21. The method of claim 20, wherein the oxidant is added in an amount of 0.15 to 5 mol, based on 1 mol of the dihydroxyphenyl moiety, the trihydroxyphenyl moiety, or the tannic acid comprised in the nanofibers.

22. The method of claim 20, wherein the metal ion is Ca²⁺, Fe²⁺, Fe3+, V⁴⁺, V³⁺ or _{V}²⁺.

23. The method of claim 20, wherein the oxidant is sodium periodate, tetrabutylammonium periodate, or hydrogen peroxide.

24. The method of claim 19, wherein a step of performing deacetylation treatment of the nanofibers is additionally performed, before the step of preparing the hydrogel.
